(19) United States
(12) Patent Application Publication
(10) Pub. No.: US 2016/0115106 A1
(43) Pub. Date: Apr. 28, 2016

(54) Stabilization of Chlorinated Olefins

(71) Applicant: Axiall Ohio, Inc., Atlanta, GA (US)

(72) Inventor: Scott A. Sherwood, Irwin, PA (US)

(21) Appl. No.: 14/887,362

(22) Filed: Oct. 20, 2015

Related U.S. Application Data
(60) Provisional application No. 62/067,085, filed on Oct. 22, 2014.

(51) Int. Cl. C07C 17/42

(52) U.S. Cl. CPC C07C 17/42

(57) ABSTRACT

A composition is provided, including: at least one chlorinated olefin having at least 3 carbon atoms and at least two terminal chlorine atoms; and at least one C₁-C₇ aliphatic aldehyde hydrazone and/or at least one C₁-C₁₄ aliphatic ketohydrazone. A method of inhibiting formation of phosgene and/or acidic impurity from a chlorinated olefin having at least 3 carbon atoms and at least two terminal chlorine atoms, comprising: adding at least one C₁-C₇ aliphatic aldehyde hydrazone and/or at least one C₁-C₁₄ aliphatic ketohydrazone to a chlorinated olefin having at least 3 carbon atoms and at least two terminal chlorine atoms also is provided.

### CROSS REFERENCE TO RELATED APPLICATIONS

The present application is entitled to and claims priority to U.S. Provisional Patent Application No. 62/067,085, filed Oct. 22, 2014, the disclosure of which is incorporated herein by reference in its entirety.

### BACKGROUND OF THE INVENTION

1. Field of the Invention

The present invention relates to compositions comprising chlorinated olefin(s) having at least 3 carbon atoms and at least two terminal chlorine atoms stabilized with C₁-C₇ aliphatic aldehyde hydrazone(s) and/or C₁-C₁₄ aliphatic ketohydrazone(s) to inhibit formation of undesirable decomposition products, such as phosgene.

2. Description of the Related Art

Halogenated hydrocarbons are useful as feedstocks for the manufacture of fluorinated hydrocarbons, such as hydrofluoroolefins (HFOs). Hydrofluoroolefins can, for example, be used as, or as components of, refrigerants, polyurethane blowing agents, fire extinguishing agents, and foaming agents. HFO's which are useful as low GWP blowing agents for thermoset and thermoplastic foams, solvents, heat transfer fluids such as in heat pumps and refrigerants include, but are not limited to, 2,3,3,3-tetrafluoropropene (1234yf), 1,3,3,3-tetrafluoropropene (1234ze), 3,3,3-trifluoropropene (1243zf), 1-chloro-3,3,3-trifluoropropene (1233zd), and 2-chloro-3,3,3 trifluoropropene (1233xf). The process of manufacturing these materials typically involves processes of fluorination with HF of chloroolefin starting materials such as 1,1,2,3-tetrachloropropene (1230xa) for manufacturing 1234yf and/or 1233xf, 1,1,3,3-tetrachloropropene (1230za) for manufacturing 1234ze and/or 1233zd and 1,1,3-trichloropropene (1240za) for manufacturing 1243zf.

However, chlorinated organic compounds, such as tetrachloropropenes, can decompose or break down in prolonged contact with heat, light, air, humidity and/or metals. Oxidation can be a major mechanism of decomposition. U.S. Pat. No. 3,959,367 discloses the oxidation products of 1,1,2,3 tetrachloropropene as including primarily 1,1,1,3-tetrachloropropanone, along with chloroacetyl chloride. Also, storage of 1,1,2,3-tetrachloropropene can create conditions which facilitate formation of tetrachloropropanone and increased acidity and phosgene (COCl₂) levels.

Phosgene is an undesirable material which can be formed during the manufacturing, processing, shipping or storage of chloroolefins. The formation of phosgene occurs by the addition of oxygen to the olefin to form the 1,2-dioxetane intermediate, followed by decomposition of the 1,2-dioxetane intermediate to carbonyl containing compounds such as phosgene and carboxylic acid chloride, as shown in Scheme 1:

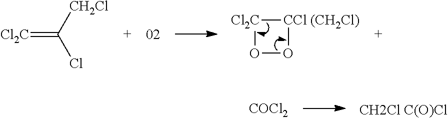

A similar degradation mechanism occurs when 1230za or 1240za or 1230xf, are stored or manufactured in ferrous containers. The formation of phosgene can occur when the precursor to chloroalkenes, such as CCl₃CHClCH₂Cl (240 db) in case of 1230xa is exposed to oxygen containing gas when stored in a ferrous container. Scheme 2 shows a typical reaction sequence:

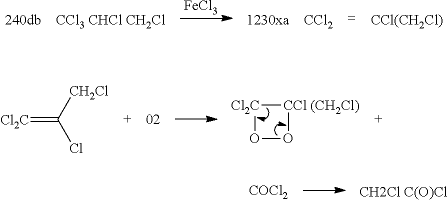

It would be desirable to inhibit or prevent the formation of phosgene and/or acids and/or acid chlorides as a result of a decomposition reaction of a chloroolefin.

### SUMMARY OF THE INVENTION

In some embodiments or features of the present invention, a composition is provided comprising: (a) at least one chlorinated olefin having at least 3 carbon atoms and at least two terminal chlorine atoms; and (b) at least one C₁-C₇ aliphatic aldehyde hydrazone and/or at least one C₁-C₁₄ aliphatic ketohydrazone.

Also provided is a method for inhibiting formation of phosgene and/or acid impurity from a chlorinated olefin having at least 3 carbon atoms and at least two terminal chlorine atoms, comprising: adding at least one C₁-C₇ aliphatic aldehyde hydrazone and/or at least one C₁-C₁₄ aliphatic ketohydrazone to a chlorinated olefin having at least 3 carbon atoms and at least two terminal chlorine atoms.

The features that characterize the present invention are pointed out with particularity in the claims, which are annexed to and form a part of this disclosure. These and other features of the invention, its operating advantages and the specific objects obtained by its use will be more fully understood from the following detailed description in which non-limiting embodiments or features of the invention are illustrated and described.

### SUMMARY OF THE INVENTION

As used herein, the singular articles “a,” “an,” and “the” include plural referents unless otherwise expressly and unequivocally limited to one referent.

Unless otherwise indicated, all ranges or ratios disclosed herein are to be understood to encompass any and all subranges or subratios subsumed therein. For example, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 6.1, 3.5 to 7.8, and 5.5 to 10.

Other than in the operating examples, or where otherwise indicated, all numbers expressing quantities of ingredients, reaction conditions, and so forth used in the specification and claims are to be understood as modified in all instances by the term “about.”

All documents, such as but not limited to issued patents and patent applications, referred to herein, and unless otherwise indicated, are to be considered to be “incorporated by reference” in their entirety.

As used herein, recitations of “alkyl” include “cycloalkyl” and/or “linear or branched alkyl.” Recitations of “linear or branched” groups, such as linear or branched alkyl, are herein understood to include: a methylene group or a methyl group; groups that are linear, such as linear C₂-C₂₅ alkyl groups; and groups that are appropriately branched, such as branched C₃-C₂₅ alkyl groups.

The term “linear or branched alkyl” as used herein, in accordance with some embodiments or features, means linear or branched C₁-C₂₅ alkyl, or linear or branched C₁-C₁₀ alkyl, or linear or branched C₂-C₁₀ alkyl. Examples of alkyl groups from which the various alkyl groups of the present invention can be selected from, include, but are not limited to, methyl, ethyl, propyl, isopropyl, butyl, isobutyl, sec-butyl, tert-butyl, pentyl, neopentyl, hexyl, heptyl, octyl, nonyl and decyl.

As used herein, recitations of “alkenyl” include “cycloalkenyl” and/or “linear or branched alkenyl” and means groups having at least one ethylenically unsaturated group, that are not aromatic. The term “alkenyl” as used herein, in accordance with some embodiments or features, includes linear or branched C₂-C₂₅ alkenyl (including, but not limited to, linear or branched C₂-C₁₀ alkenyl). Examples of alkenyl groups include but are not limited to vinyl, allyl, propenyl, butenyl, pentenyl, and hexenyl.

As used herein, the term “aryl” includes cyclic aryl groups and polycyclic aryl groups. With some embodiments or features, aryl groups include, but are not limited to, C₆-C₈ aryl, such as C₆-C₁₀ aryl (including polycyclic aryl groups). Examples of aryl groups include, but are not limited to, phenyl, naphthyl, anthracenyl and triptycenyl.

As used herein, recitations of “alkene” include “cycloalkene” and/or “linear or branched alkene” and means hydrocarbons having at least one ethylenically unsaturated group, that are not aromatic. The term “linear or branched alkene” as used herein, in accordance with some embodiments or features, means linear or branched C₂-C₂₅ alkene (including, but not limited to, linear or branched C₂-C₁₀ alkene). Examples of alkenes include, but are not limited to, ethene, propene, butene, pentene, hexene, heptene, octane, nonene, and decene.

As used herein, the term “aromatic,” such as aromatic compound, includes cyclic aromatic and polycyclic aromatic. With some embodiments or features, aromatic compounds include, but are not limited to, C₆-C₁₈ aromatic compounds, such as C₆-C₁₀ aromatic compounds (including polycyclic aromatic compounds). Examples of aromatic compounds include, but are not limited to, benzene, naphthalene, anthracene and triptycene.

The compositions of the present invention comprise at least one (one or more) chlorinated olefin(s) or alkenes having at least 3 carbon atoms and at least two terminal chlorine atoms, such as chlorinated propene, chlorinated butenes, chlorinated pentenes, chlorinated hexenes, etc.

Suitable chlorinated propenes include compounds having two, three, four, five, or six chlorine atoms, referred to as di-, tri-, tetra-, penta-, or hexa-chloropropenes respectively. Non-limiting examples of chlorinated propenes include dichloropropenes, such as 1,1,-dichloropropene; trichloropropenes, such as 1,1,2-trichloropropene, 1,1,3-trichloropropene (1240za), E-1,2,3-trichloropropene, Z-1,2,3-trichloropropene, E-1,3,3-trichloropropene, Z-1,3,3-trichloropropene, 2,3,3-trichloropropene, and 3,3,3-trichloropropene, tetrachloropropenes, such as 1,1,2,3-tetrachloropropene (1230xa), 1,1,3,3-tetrachloropropene (1230za), 1,1,1,2-tetrachloropropene (1230xl), E-1,2,3,3-tetrachloropropene, Z-1,2,3,3-tetrachloropropene, E-1,3,3,3-tetrachloropropene, Z-1,3,3,3-tetrachloropropene, and 2,3,3,3-tetrachloropropene, pentachloropropenes, such as 1,1,2,3,3-pentachloropropene, 1,1,3,3,3-pentachloropropene, E-1,2,3,3,3-pentachloropropene, and Z-1,2,3,3,3-pentachloropropene, and hexachloropropene, any of which, or mixtures of any of which, may be used in the compositions described herein. In some embodiments or features, the chlorinated propene is a tetrachloropropene, for example 1,1,2,3-tetrachloropropene or 2,3,3,3-tetrachloropropene or 1,1,3,3-tetrachloropropene or 1,3,3,3-tetrachloropropene or (cis or trans)-1,2,3,3-tetrachloropropene. Preferred tetrachloropropenes for use in the compositions of the invention include 1,1,2,3-tetrachloropropene, 2,3,3,3-tetrachloropropene and 1,1,3,3-tetrachloropropene.

In some embodiments or features, the amount of chlorinated olefin(s) can range from about 1 to about 100 weight percent, or about 20 to about 100 weight percent, or about 50 to about 100 weight percent, or about 90 to about 100 weight percent, on a basis of total weight of the components of the composition.

The present invention provides stable, purified chloroolefin compositions which are particularly useful for the manufacture of HFOs and HCFOs such as 1234yf, 1233xf, 1234ze, 1233zd and 1243zf.

As discussed above, the compositions according to the present invention comprise at least one (one or more) C₁-C₇ aliphatic aldehyde hydrazone(s) and/or at least one (one or more) C₁-C₁₄ aliphatic ketohydrazone(s). While not intending to be bound by any theory, it is believed that the aliphatic aldehyde hydrazone and/or aliphatic ketohydrazone has a double carbon bond that is more highly reactive than the double carbon bond in the chlorinated olefin, thereby reducing the likelihood of forming decomposition products from the chlorinated olefin when the olefin is exposed to conditions such as heat, light, air, humidity and/or metals favoring formation of undesired byproducts, such as phosgene; phosgene precursors, such as substituted 2,2-dichlorooxiranes (epoxides), such as those of the formula:

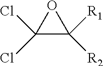

wherein R₁=H, Cl, C₁-C₈, and R₂=C₁-C₈; and/or substituted 4,4-dioxetanes (peroxides) such as those of the formula:

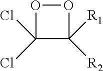

wherein R₁=H, Cl, C₁-C₈, and R₂=C₁-C₈.

The aliphatic aldehyde hydrazone(s) used in the compositions of the present invention may be prepared by condensing an aliphatic aldehyde, notably aldehydes having from 1 to 8 carbon atoms such as formaldehyde, acetaldehyde, propionaldehyde, acrolein, chloral and dichloroacetaldehyde, with hydrazine or a substituted hydrazine.

The aliphatic ketohydrazone(s) used in the compositions of the present invention may be prepared by condensing an aliphatic ketone, notably ketones having from 1 to 14 carbon atoms such as acetone, methyl ethyl ketone (MEK) (2-butanone), diethyl ketone (3-pentanone), with hydrazine or a substituted hydrazine.

In some embodiments or features, the hydrazine may be represented by structural formula I:

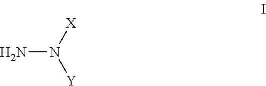

wherein X and Y are each hydrogen or alkyl groups having 1 to 8 carbons, e.g., dimethyl hydrazine, diethyl hydrazine, methyl hydrazine, ethyl hydrazine, methyl ethyl hydrazine and propyl methyl hydrazine.

Preferably, the aliphatic aldehyde hydrazones used are those having a total of between 1 and 7 carbons, with no aliphatic group having more than 4 carbon atoms linked to the aldehyde hydrazone characterizing structure, which may be represented by structural formula II:

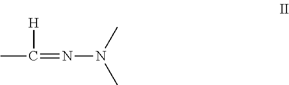

The aliphatic aldehyde hydrazones that may be used in accordance with the present invention may be represented by structural formula III:

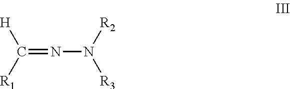

wherein each of R₁, R₂ and R₃ may be hydrogen or an aliphatic group (including saturated and unsaturated aliphatic groups) of from 1 to 4 carbons, with the proviso that the aliphatic aldehyde hydrazone has a total of from 1 to 7 carbon atoms in the aliphatic groups, R₁, R₂ and R₃. For most of the aliphatic aldehyde hydrazones, the sum of the carbon atoms in the groups represented by R₁, R₂ and R₃ is not more than 5. Often the aliphatic groups of the aliphatic aldehyde are alkyl groups. Aliphatic aldehyde hydrazones are described in U.S. Pat. Nos. 3,043,888, 4,026,956 and 4,418,231, the disclosures of which are incorporated herein by reference.

Non-limiting examples of aliphatic aldehyde hydrazones include formaldehyde hydrazone, formaldehyde dimethylhydrazone, formaldehyde diethyl hydrazone, formaldehyde methyl ethyl hydrazone, acetaldehyde methylhydrazone, (E)-acetaldehyde methylhydrazone, (Z)-acetaldehyde methylhydrazone, acetaldehyde dimethyl hydrazone (2-Ethylidene-1,1-dimethylhydrazine), (E)-acetaldehyde dimethyl hydrazone, (Z)-acetaldehyde dimethyl hydrazone, acetaldehyde methyl ethyl hydrazone, (E)-acetaldehyde methyl ethyl hydrazone, (Z)-acetaldehyde methyl ethyl hydrazone, formaldehyde propylhydrazone, formaldehyde isopropyl hydrazone, propionaldehyde hydrazone, (E)-propionaldehyde hydrazone, (Z)-propionaldehyde hydrazone, and mixtures thereof. In some embodiments or features, the aliphatic aldehyde hydrazone is selected from acetaldehyde dimethyl hydrazone, acetaldehyde methyl ethyl hydrazone or mixtures thereof, and more preferably, is acetaldehyde dimethyl hydrazone.

Non-limiting examples of aliphatic ketohydrazones include acetone hydrazone, 3-pentanone hydrazone, butyrone hydrazone, 5-nonanone hydrazone, 6-undecanone hydrazone, 2-butanone hydrazone, (2E)-2-butanone hydrazone, (2Z)-2-butanone hydrazone, 2-pentanone hydrazone, (2E)-2-pentanone hydrazone, (2Z)-2-pentanone hydrazone, E-hexanone hydrazone, Z-hexanone hydrazone, 3-hexanone hydrazone, (3E)-3-hexanone hydrazone, (3Z)-3-hexanone hydrazone, 3-methyl-4-heptanone hydrazone, (4E)-3-methyl-4-heptanone hydrazone, (4Z)-3-methyl-4-heptanone hydrazone, 2,4-dimethyl-3-pentanone hydrazone, 2,2,4,4-tetramethyl-3-pentanone hydrazone, 2,2,6,6-tetramethyl-4-heptanone hydrazone, 2-heptanone hydrazone, (2E)-2-heptanone hydrazone, (2Z)-2-heptanone hydrazone, etc.

The aliphatic aldehyde hydrazones and aliphatic ketohydrazones of the present invention incorporate a double bond, and, therefore, may exist in different stereoisomeric forms. It is intended that all geometric isomers of the aliphatic aldehyde hydrazones and aliphatic ketohydrazones of the present invention as well as mixtures thereof, including racemic mixtures, form part of the present invention. For example, both the cis- and trans-forms or both the E and Z isomers, as well as mixtures, are embraced within the scope of the invention.

The amount of C₁-C₇ aliphatic aldehyde hydrazone(s) and/or C₁-C₁₄ aliphatic ketohydrazone(s) present in the compositions of the present invention is a storage stabilizing amount, i.e., an amount sufficient to substantially inhibit formation of phosgene or other oxiranes (epoxides) or dioxetanes (peroxides) during storage. The time for storage may be a short period of a few weeks or few months or a longer period of up to several years. The amount of C₁-C₇ aliphatic aldehyde hydrazone(s) and/or C₁-C₁₄ aliphatic ketohydrazone(s) may range from at least about 1 part per million parts of the composition (ppm), or at least about 5 ppm, or at least about 10 ppm, or at least about 15 ppm. The amount of C₁-C₇ aliphatic aldehyde hydrazone(s) and/or C₁-C₁₄ aliphatic ketohydrazone(s) is usually less than about 10,000 ppm, or less than about 1000 ppm, or not more than about 100 ppm, or not more than about 75 ppm, or not more than about 50 ppm, or not more than about 25 ppm, or about at least about 1 part per million to less than about 1000 ppm on a basis of total components of the composition. In some embodiments or features, the amount of C₁-C₇ aliphatic aldehyde hydrazone(s) and/or C₁-C₁₄ aliphatic ketohydrazone(s) in the composition ranges from about 1 part per million parts of the composition (ppm) to about 100 ppm on a basis of total components of the composition. The amount of C₁-C₇ aliphatic aldehyde hydrazone(s) and/or C₁-C₁₄ aliphatic ketohydrazone(s) used may range between any combination of these values, inclusive of the recited values.

In some embodiments or features, the compositions can further comprise at least one (one or more) free radical inhibitors, for example inhibitors of free-radical reactions such as those commonly used to prevent free-radical oxidations, including:

hydroxy-substituted aromatic compounds such as phenolic compounds (for example isopropyl-meta cresol (thymol), 4-tertiary-amyl phenol, 2,6-di-t-butylphenol, 4,4′-methylenebis(2,6-di-tert-butyl-phenol, tocopherol) and naphthol compounds (for example 4-methoxy-1-naphthol and 2-, 3-, 5-, 6-, 7-, or 8-alkyl substituted derivatives or combinations thereof, e.g., 6,7-dimethyl-4-methoxy-1-naphthol); 4,4′-bis(2,6-di-tert-butylphenol, 2,2-biphenyldiols, 4,4-biphenyldiols; derivatives of 2,2- and 4,4-biphenyldiols; 2,2′-methylenebis(4-ethyl-6-tert-butylphenol); 2,2′-methylenebis(4-methyl-6-tertbutylphenol); 4,4,-butylidenebis (3-methyl-6-tert-butylphenol); 4,4-isopropylidenebis(2,6-di-tert-butylphenol); 2,2′-methylenebis(4-methyl-6-nonylphenol); 2,2′-isobutylidenebis(4,6-dimethylphenol); 2,2′-methylenebis(4-methyl-6-cyclohexylphenol); 2,6-di-tert-butyl-4-methyl-phenol; 2,6-di-tert-butyl-4-ethylphenol; 2,4-dimethyl-6-tert-butylphenol; 2,6-di-tert-butyl-4-(N,N′-dimethylaminomethyl)phenol; 4,4′-thiobis(2-methyl-6-tertbutylphenol); 4,4′-thiobis(3-methyl-6-tert-butylphenol); 2,2′-thiobis(4-methyl-6-tertbutylphenol);

alkoxy-substituted aromatic compounds such as alkoxyphenols (4-methoxyphenol (HQMME), 3-methoxyphenol, 2-methoxy-4-(1-propenyl)phenol (isoeugenol), butylated for example hydroxy anisole (BHA) such as 2-tert-butyl-4-hydroxyanisole and 3-tert-butyl-4-hydroxyanisole);

phenol compounds substituted with an allyl group, for example as o-allylphenol, m-allylphenol, p-allylphenol, 4-allyl-2-methoxyphenol (eugenol);

hydroquinones, for example as methoxy-hydroquinone and tert-butyl hydroquinone;

and mixtures thereof.

The amount of free radical inhibitor(s) can range from about 1 ppm to about 50,000 ppm, or about 1 ppm to about 10,000 ppm, or about 1 ppm to about 1,000 ppm, or about 1 to about 100 ppm, based upon weight of the halogenated alkene substrate.

In some embodiments or features, the compositions can further comprise at least one (one or more) acid scavengers, such as 1,2-butylene oxide, etc.

### EXAMPLES

### Examples 1-4

Samples of HCO-1230xa, HCO-1230xa spiked with diisopropylamine, and HCO-1230xa spiked with a solution of acetaldehyde dimethylhydrazone (ADH) dissolved in trans-1,2-dichloroethylene were tested for stability for 30 days at room temperature (˜20° C.) without shielding from light. Colorless 10-mL glass vials with PTFE-faced silicone septa were used as the sealed containers. Approximately 5 grams of sample was placed in each vial (˜3.33 mL, ˜2:1 headspace to liquid ratio). Total acidity (as ppm hydrogen chloride) was determined by sample titration with sodium hydroxide (0.01N NaOH in methanol) using a bromothymol blue endpoint. Analysis of phosgene was determined by the use of phosgene detection tape (DOD Technologies, Inc.) in the headspace of the opened vials.

Sample preparations:

1) HCO-1230xa—4.98 grams (control)

2) HCO-1230xa and diisopropylamine—4,9689 grams HCO-1230xa+0.0011 grams diisopropylamine (˜223 ppm diisopropylamine concentration)

3) HCO-1230xa and ADH—5.2784 grams HCO-1230xa+0.0016 grams 12 wt % ADH in trans-1,2-dichloroethylene (˜37 ppm ADH concentration)

The following Table 1 provides a summary of the stability test results of Examples 1-4.

TABLE 1

| | | | Total |
|---|---|---|---|
| | | | Acidity |
| Ex | Sample | Phosgene, tape color | (ppm HCl) |
| 1 | HCO-1230xa (Day 0) | Light yellow (−test) | 124 |
| 2 | HCO-1230xa (Day 30) | Dark red (+test) | 844 |
| 3 | HCO-1230xa and | Dark red (+test) | 1263 |
| | diisopropylamine (Day 30) | | |
| 4 | HCO-1230xa and ADH (Day 30) | Light yellow (−test) | 140 |

### Examples 5-8

Examples 5-8 demonstrate short-term stabilization effects of the present invention. Approximately 5 grams of 1,1,2,3-tetrachloropropene containing approximately 0.015 area % of phosgene precursor and a pre-determined amount of stabilizer was charged to a 10-mL glass gas chromatography headspace vial. The vial was capped (perfluoroethylene-faced silicone septum) in an air atmosphere and allowed to stand undisturbed at room temperature for the desired testing period (7-61 days). The vial was uncapped and immediately tested with phosgene detection tape (DOD Technologies, Inc., Part number: 1-200-006). The tape was placed in the headspace of the vial for 30 seconds to determine if phosgene was present. A sample of the vial liquid was titrated for acidity content with 0.01N sodium hydroxide in methanol to a bromothymol blue endpoint. The following Table 2 provides a summary of the stability test results of Examples 5-8 in comparison to Example 4 discussed above.

TABLE 2

| | | | | Acidity | Acidity | |
|---|---|---|---|---|---|---|
| | | | Active | Before | after | Presence |
| | | Test | Stabi- | Testing | Testing | of Free |
| | | Period | lizer(s) | (as ppm | (as ppm | Phosgene |
| Ex | Stabilizer | (days) | (ppm) | HCl) | HCl) | (+/−) |
| 5 | Control | 7 | 0 | 124 | 668 | + |
| 6 | Diisopropylamine | 7 | 173 | 160 | 758 | + |
| 7 | 4-Methoxyphenol | 61 | 309 | 118 | 113 | + |
| 8 | 4-t-Amylphenol | 61 | 171 | 115 | 116 | + |
| 4 | Acetaldehyde | 30 | 37 | 118 | 140 | − |
| | Dimethyl- | | | | | |
| | hydrazone* | | | | | |

*Supplied as 12 wt % acetaldehyde dimethylhydrazone in trans-1,2-dichloroethylene

### Examples 9-14

Examples 9-14 demonstrate long-term stabilization effects of the present invention. Approximately 10 grams of 1,1,2,3-tetrachloropropene containing approximately 0.035 area % of phosgene precursor and a pre-determined amount of stabilizer was charged to a 20-mL glass gas chromatography headspace vial. The vial was capped (perfluroroethylene-faced silicone septum) in an air atmosphere and allowed to stand undisturbed at room temperature for the desired testing period (7-189 days). The vial was uncapped and immediately tested with phosgene detection tape (DOD Technologies, Inc., Part number: 1-200-006). The tape was placed in the headspace of the vial for 60 seconds to determine if phosgene was present. A sample of the vial liquid was titrated for acidity content with 0.01N sodium hydroxide in methanol to a bromothymol blue endpoint. The following Table 3 provides a summary of the stability test results of Examples 9-14.

TABLE 3

| | | | | Acidity | | |
|---|---|---|---|---|---|---|
| | | | | Before | Acidity after | Presence |
| | | Test | Active | Testing, | Testing, | of Free |
| | | Period, | Stabilizer(s), | as ppm | as ppm | Phosgene, |
| Ex | Stabilizer | Days | ppm | HCl | HCl | +/− |
| 9 | Acetaldehyde | 112 | 31 | 77 | 132 | − |
| | Dimethylhydrazone* | | | | | |
| 10 | Acetaldehyde | 56 | 15 | 77 | 102 | − |
| | Dimethylhydrazone* | | | | | |
| 11 | 4-Methoxyphenol + | 189 | 31 + 25 | 73 | 160 | − |
| | Acetaldehyde | | | | | |
| | Dimethylhydrazone* | | | | | |
| 12 | 4-Methoxyphenol + | 189 | 13 + 15 | 91 | 145 | − |
| | Acetaldehyde | | | | | |
| | Dimethylhydrazone* | | | | | |
| 13 | 4-t-Amylphenol + | 189 | 34 + 32 | 84 | 149 | − |
| | Acetaldehyde | | | | | |
| | Dimethylhydrazone* | | | | | |
| 14 | 4-t-Amylphenol + | 189 | 23 + 15 | 87 | 133 | − |
| | Acetaldehyde | | | | | |
| | Dimethylhydrazone* | | | | | |

*Supplied as 12 wt % acetaldehyde dimethylhydrazone in trans-1,2-dichloroethylene

### Comparative Examples 15-20

The procedure of Examples 9-14 was followed. The following Table 4 provides a summary of the stability test results of Examples 15-20.

TABLE 4

| | | | | Acidity | Acidity | |
|---|---|---|---|---|---|---|
| | | | Active | Before | after | Presence |
| | | Test | Stabi- | Testing | Testing | of Free |
| | | Period | lizer(s) | (as ppm | (as ppm | Phosgene |
| Ex | Stabilizer | (days) | (ppm) | HCl) | HCl) | (+/−) |
| 15 | 4-Methoxyphenol | 189 | 30 | 101 | 138 | + |
| 16 | 4-Methoxyphenol | 189 | 15 | 77 | 142 | + |
| 17 | 4-t-Amylphenol | 189 | 30 | 88 | 119 | + |
| 18 | 4-t-Amylphenol | 189 | 15 | 94 | 144 | + |
| 19 | Triethylamine | 7 | 30 | 78 | 921 | + |
| 20 | Triethylamine | 7 | 2 | 99 | 623 | + |

The preceding examples demonstrate some of the unexpected and desirable results that can be obtained with the method of the present invention, such as, but not limited to, the inhibition of phosgene formation and acid formation in a sample of HCO-1230xa using ADH according to the present invention.

The present invention has been described with reference to specific details of particular embodiments or features thereof. However, it is not intended that such details be regarded as limitations upon the scope of the invention except insofar as and to the extent that they are included in the accompanying claims.

The present invention is also directed to the following clauses.

Clause 1: A composition comprising: (a) at least one chlorinated olefin having at least 3 carbon atoms and at least two terminal chlorine atoms; and (b) at least one C₁-C₇ aliphatic aldehyde hydrazone and/or at least one C₁-C₁₄ aliphatic ketohydrazone.

Clause 2: The composition according to clause 1, wherein the at least one chlorinated olefin comprises at least one chlorinated propene.

Clause 3: The composition according to any of clauses 1 or 2, wherein the at least one chlorinated propene comprises at least one trichloropropene selected from the group consisting of 1,1,2-trichloropropene, 1,1,3-trichloropropene (1240za), E-1,2,3-trichloropropene, Z-1,2,3-trichloropropene, E-1,3,3-trichloropropene, Z-1,3,3-trichloropropene, 2,3,3-trichloropropene, and 3,3,3-trichloropropene.

Clause 4: The composition according to any of clauses 1 or 2, wherein the at least one chlorinated propene comprises at least one tetrachloropropene selected from the group consisting of 1,1,2,3-tetrachloropropene (1230xa), 1,1,3,3-tetrachloropropene (1230za), 1,1,1,2-tetrachloropropene (1230xl), E-1,2,3,3-tetrachloropropene, Z-1,2,3,3-tetrachloropropene, E-1,3,3,3-tetrachloropropene, Z-1,3,3,3-tetrachloropropene, and 2,3,3,3-tetrachloropropene.

Clause 5: The composition according to any of clauses 1 or 2, wherein the at least one chlorinated propene comprises 1,1,2,3-tetrachloropropene (1230xa).

Clause 6: The composition according to any of clauses 1 or 2, wherein the at least one chlorinated propene comprises at least one pentachloropropene selected from the group consisting of 1,1,2,3,3-pentachloropropene, 1,1,3,3,3-pentachloropropene, E-1,2,3,3,3-pentachloropropene, and Z-1,2,3,3,3-pentachloropropene.

Clause 7: The composition according to any of clauses 1 or 2, wherein the at least one chlorinated propene comprises hexachloropropene.

Clause 8: The composition according to any of clauses 1-7, wherein the amount of the at least one chlorinated olefin(s) can range from about 1 to about 100 weight percent.

Clause 9: The composition according to any of clauses 1-8, wherein the at least one C₁-C₇ aliphatic aldehyde hydrazone(s) is represented by structural formula III:

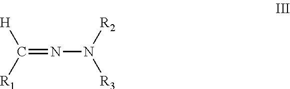

wherein each of R₁, R₂ and R₃ is hydrogen or a saturated or unsaturated aliphatic group of from 1 to 4 carbons, with the proviso that the aliphatic aldehyde hydrazone has a total of from 1 to 7 carbon atoms in the aliphatic groups, R₁, R₂ and R₃.

Clause 10: The composition according to any of clauses 1-9, wherein the at least one C₁-C₇ aliphatic aldehyde hydrazone is selected from the group consisting of formaldehyde hydrazone, formaldehyde dimethylhydrazone, formaldehyde diethyl hydrazone, formaldehyde methyl ethyl hydrazone, acetaldehyde methylhydrazone, (E)-acetaldehyde methylhydrazone, (Z)-acetaldehyde methylhydrazone, acetaldehyde dimethyl hydrazone (2-Ethylidene-1,1-dimethylhydrazine), (E)-acetaldehyde dimethyl hydrazone, (Z)-acetaldehyde dimethyl hydrazone, acetaldehyde methyl ethyl hydrazone, (E)-acetaldehyde methyl ethyl hydrazone, (Z)-acetaldehyde methyl ethyl hydrazone, formaldehyde propylhydrazone, formaldehyde isopropyl hydrazone, propionaldehyde hydrazone, (E)-propionaldehyde hydrazone, (Z)-propionaldehyde hydrazone, and mixtures thereof.

Clause 11: The composition according to any of clauses 1-10, wherein the at least one C₁-C₇ aliphatic aldehyde hydrazone comprises acetaldehyde dimethyl hydrazone, isomers, and mixtures thereof.

Clause 12: The composition according to any of clauses 1-8, wherein the at least one C₁-C₁₄ aliphatic ketohydrazone include acetone hydrazone, 3-pentanone hydrazone, butyrone hydrazone, 5-nonanone hydrazone, 6-undecanone hydrazone, 2-butanone hydrazone, (2E)-2-butanone hydrazone, (2Z)-2-butanone hydrazone, 2-pentanone hydrazone, (2E)-2-pentanone hydrazone, (2Z)-2-pentanone hydrazone, E-hexanone hydrazone, Z-hexanone hydrazone, 3-hexanone hydrazone, (3E)-3-hexanone hydrazone, (3Z)-3-hexanone hydrazone, 3-methyl-4-heptanone hydrazone, (4E)-3-methyl-4-heptanone hydrazone, (4Z)-3-methyl-4-heptanone hydrazone, 2,4-dimethyl-3-pentanone hydrazone, 2,2,4,4-tetramethyl-3-pentanone hydrazone, 2,2,6,6-tetramethyl-4-heptanone hydrazone, 2-heptanone hydrazone, (2E)-2-heptanone hydrazone, (2Z)-2-heptanone hydrazone, and mixtures thereof.

Clause 13: The composition according to any of clauses 1-12, wherein the amount of C₁-C₇ aliphatic aldehyde hydrazone(s) and/or at least one C₁-C₁₄ aliphatic ketohydrazone(s) in the composition ranges from about 1 part per million parts of the composition (ppm) to about 10,000 ppm on a basis of total components of the composition.

Clause 14: A method for inhibiting formation of phosgene and/or acidic impurity from a chlorinated olefin having at least 3 carbon atoms and at least two terminal chlorine atoms, comprising: adding at least one C₁-C₇ aliphatic aldehyde hydrazone and/or at least one C₁-C₁₄ aliphatic ketohydrazone to a chlorinated olefin having at least 3 carbon atoms and at least two terminal chlorine atoms.

The invention claimed is:

1. A composition comprising:
(a) at least one chlorinated olefin having at least 3 carbon atoms and at least two terminal chlorine atoms; and
(b) at least one C₁-C₇ aliphatic aldehyde hydrazone and/or at least one C₁-C₁₄ aliphatic ketohydrazone.

2. The composition according to claim 1, wherein the at least one chlorinated olefin comprises at least one chlorinated propene.

3. The composition according to claim 1, wherein the at least one chlorinated propene comprises at least one trichloropropene selected from the group consisting of 1,1,2-trichloropropene, 1,1,3-trichloropropene (1240za), E-1,2,3-trichloropropene, Z-1,2,3-trichloropropene, E-1,3,3-trichloropropene, Z-1,3,3-trichloropropene, 2,3,3-trichloropropene, and 3,3,3-trichloropropene.

4. The composition according to claim 1, wherein the at least one chlorinated propene comprises at least one tetrachloropropene selected from the group consisting of 1,1,2,3-tetrachloropropene (1230xa), 1,1,3,3-tetrachloropropene (1230za), 1,1,1,2-tetrachloropropene (1230xl), E-1,2,3,3-tetrachloropropene, Z-1,2,3,3-tetrachloropropene, E-1,3,3,3-tetrachloropropene, Z-1,3,3,3-tetrachloropropene, and 2,3,3,3-tetrachloropropene.

5. The composition according to claim 1, wherein the at least one chlorinated propene comprises 1,1,2,3-tetrachloropropene (1230xa).

6. The composition according to claim 1, wherein the at least one chlorinated propene comprises at least one pentachloropropene selected from the group consisting of 1,1,2,3,3-pentachloropropene, 1,1,3,3,3-pentachloropropene, E-1,2,3,3,3-pentachloropropene, and Z-1,2,3,3,3-pentachloropropene.

7. The composition according to claim 1, wherein the at least one chlorinated propene comprises hexachloropropene.

8. The composition according to claim 1, wherein the amount of the at least one chlorinated olefin(s) can range from about 1 to about 100 weight percent.

9. The composition according to claim 1, wherein the at least one C₁-C₇ aliphatic aldehyde hydrazone(s) is represented by structural formula III: 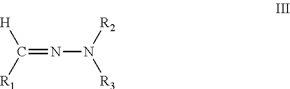
wherein each of R₁, R₂ and R₃ is hydrogen or a saturated or unsaturated aliphatic group of from 1 to 4 carbons, with the proviso that the aliphatic aldehyde hydrazone has a total of from 1 to 7 carbon atoms in the aliphatic groups, R₁, R₂ and R₃.

10. The composition according to claim 1, wherein the at least one C₁-C₇ aliphatic aldehyde hydrazone is selected from the group consisting of formaldehyde hydrazone, formaldehyde dimethylhydrazone, formaldehyde diethyl hydrazone, formaldehyde methyl ethyl hydrazone, acetaldehyde methylhydrazone, (E)-acetaldehyde methylhydrazone, (Z)-acetaldehyde methylhydrazone, acetaldehyde dimethyl hydrazone (2-Ethylidene-1,1-dimethylhydrazine), (E)-acetaldehyde dimethyl hydrazone, (Z)-acetaldehyde dimethyl hydrazone, acetaldehyde methyl ethyl hydrazone, (E)-acetaldehyde methyl ethyl hydrazone, (Z)-acetaldehyde methyl ethyl hydrazone, formaldehyde propylhydrazone, formaldehyde isopropyl hydrazone, propionaldehyde hydrazone, (E)-propionaldehyde hydrazone, (Z)-propionaldehyde hydrazone, and mixtures thereof.

11. The composition according to claim 1, wherein the at least one C₁-C₇ aliphatic aldehyde hydrazone comprises acetaldehyde dimethyl hydrazone, isomers, and mixtures thereof.

12. The composition according to claim 1, wherein the at least one C₁-C₁₄ aliphatic ketohydrazone include acetone hydrazone, 3-pentanone hydrazone, butyrone hydrazone, 5-nonanone hydrazone, 6-undecanone hydrazone, 2-butanone hydrazone, (2E)-2-butanone hydrazone, (2Z)-2-butanone hydrazone, 2-pentanone hydrazone, (2E)-2-pentanone hydrazone, (2Z)-2-pentanone hydrazone, E-hexanone hydrazone, Z-hexanone hydrazone, 3-hexanone hydrazone, (3E)-3-hexanone hydrazone, (3 Z)-3-hexanone hydrazone, 3-methyl-4-heptanone hydrazone, (4E)-3-methyl-4-heptanone hydrazone, (4Z)-3-methyl-4-heptanone hydrazone, 2,4-dimethyl-3-pentanone hydrazone, 2,2,4,4-tetramethyl-3-pentanone hydrazone, 2,2,6,6-tetramethyl-4-heptanone hydrazone, 2-heptanone hydrazone, (2E)-2-heptanone hydrazone, (2Z)-2-heptanone hydrazone, and mixtures thereof.

13. The composition according to claim 1, wherein the amount of C₁-C₇ aliphatic aldehyde hydrazone(s) and/or at least one C₁-C₁₄ aliphatic ketohydrazone(s) in the composition ranges from about 1 part per million parts of the composition (ppm) to about 10,000 ppm on a basis of total components of the composition.

14. A method for inhibiting formation of phosgene and/or acidic impurity from a chlorinated olefin having at least 3 carbon atoms and at least two terminal chlorine atoms, comprising: adding at least one C₁-C₇ aliphatic aldehyde hydrazone and/or at least one C₁-C₁₄ aliphatic ketohydrazone to a chlorinated olefin having at least 3 carbon atoms and at least two terminal chlorine atoms.
